(19) United States
(12) Patent Application Publication
(10) Pub. No.: US 2024/0415766 A1
(43) Pub. Date: Dec. 19, 2024

(54) Preparation method of voriconazole hydrogel and eye drops using the same

(71) Applicant: EYE INSTITUTE OF SHANDONG FIRST MEDICAL UNIVERSITY (SHANDONG EYE INSTITUTE, QINGDAO EYE HOSPITAL, Qingdao (CN)

(72) Inventors: Lixin Xie, Qingdao (CN); Depeng Shi, Qingdao (CN); Hongwei Wang, Qingdao (CN)

(21) Appl. No.: 18/735,127

(22) Filed: Jun. 5, 2024

(51) Int. Cl. A61K 9/06; A61K 9/00; A61K 31/506; A61K 47/02; A61K 47/34; A61K 47/36; A61P 27/02; A61P 31/10

(52) U.S. Cl. CPC A61K 9/06; A61K 9/0048; A61K 31/506; A61K 47/02; A61K 47/34; A61K 47/36; A61P 27/02; A61P 31/10

(57) ABSTRACT

In response to poor solubility and permeability of conventional voriconazole eye drops and short retention time thereof at the ocular surface, a preparation method of voriconazole hydrogel and eye drops using the same are provided. The preparation method includes steps of: adding DAPA and sodium hydroxide to deionized water, stirring until completely dissolved to produce a DAPA solution; dropping the DAPA solution into triformylbenzene ethanol solution, and reacting at room temperature; then centrifugating, washing precipitation with ethanol, and freeze-drying in vacuum, so as to obtain a polyaldehyde oligomer; and reacting the polyaldehyde oligomer with AHA to synthesize the voriconazole hydrogel. A preparation method of a topical ocular drug includes: applying a therapeutically effective amount the voriconazole hydrogel, wherein the topical ocular drug is for mechanical injury or fungal keratitis. The present invention improves aqueous solubility, penetration, and ocular surface retention time of VCZ.

### CROSS REFERENCE OF RELATED APPLICATION

The present invention claims priority under 35 U.S.C. 119(a-d) to CN 202311691638.X, filed Dec. 11, 2023.

### BACKGROUND OF THE PRESENT INVENTION

### Field of Invention

The present invention relates to a technical field of ocular topical drugs, and more particularly to a preparation method of voriconazole hydrogel and eye drops using the same.

### Description of Related Arts

Fungal keratitis is a serious fungal infection of the eye and is one of the leading causes of infectious corneal blindness. Currently, topical eye drops such as natamycin, voriconazole (VCZ), amphotericin B, and other antifungal drugs are the main therapeutic agents for fungal keratitis. However, the unique physiological anatomy of ocular tissues limits the residence time and permeability of these drugs, resulting in low bioavailability. Frequent dosing, although necessary to maintain effective drug concentrations, can lead to various side effects and promote antifungal resistance. In the case of voriconazole, for example, voriconazole ocular surface drops are poorly and irregularly absorbed, requiring repeated administration. The water solubility of voriconazole is low, and cyclodextrin is commonly used as a solubilizing agent to increase the solubility of voriconazole. However, voriconazole aqueous solution still cannot be stored stably for a long period of time. In addition, the osmolality of voriconazole eye drops is low and irritating, and osmolality regulators such as sodium chloride, glycerol, propylene glycol, and mannitol are commonly used to regulate it. Chinese patent CN113509436A disclosed a preparation method of eye drops, which adopts sulfobutyl-β-cyclodextrin as a solubilizer, uses HCl and NaOH as pH adjusters, uses NaCl as an osmolality regulator, and adds a viscosity agent and a bacteriostatic agent. Chinese patent CN116570558A disclosed a voriconazole ophthalmic nano-sustained-release composition, comprising: voriconazole and/or a pharmaceutically usable salt thereof, a solubilizer (said solubilizer is hydroxypropyl-β-cyclodextrin, sulfobutyl-β-cyclodextrin, or a combination thereof), and hydroxypropyl methylcellulose. The aqueous hydroxypropyl methylcellulose solution has a certain degree of viscosity, which can prolong the drug's retention time in the eye, thereby achieving a sustained-release effect and reduce the number of medications. The average particle size of the controlled encapsulants was less than 20 nm, D90<100 nm, with a certain viscosity, which could properly adhere to the ocular surface and resist the tear film barrier. At the same time, due to the small particle size, the encapsulants can pass through the corneal barrier and be released slowly at the ocular surface, which ultimately presents sustained inhibition of the fungus. Application of conventional cyclodextrins for solubilization can increase the viscosity of the drug, but eye medication still needs to be 12 times per day during the first week. Meanwhile, the concentration of voriconazole is 1%, which cannot improve the permeability.

Nano-enzymatic composite hydrogels are biocomposites consisting of nano-enzymes (a nanomaterial with enzymatic activity) and hydrogels (a highly hydrated polymer network). In ophthalmic applications, this complex shows great potential with its excellent biocompatibility and tunability. The biocompatibility can prevent irritation or damage from ocular tissues, while the tunability allows for customized solutions based on the needs of specific ocular diseases.

Chinese patent CN115300459A disclosed a method for preparing a nano-enzymatic composite hydrogel eye drop, which synthesizing tannic coordinated silver-cobalt composite nanoparticles (TCN) through a coordination reaction between cobalt chloride hexahydrate and ammonia as well as a redox reaction between silver nitrate and tannic acid; using water as a medium, mixing the TCN and acrylate modified gelatin, and ultrasonicating until homogeneous; and then preparing a tannic nano-enzymatic composite hydrogel material (TCNH) by UV irradiation; and then obtaining TCNH eye drops by mixing TCNH with hydrogen peroxide solution. The material has no ability to solubilize voriconazole, and cannot be loaded with voriconazole.

Chinese patent CN108434093A disclosed a method for preparing an injectable hydrogel for intraocular drug controlled-release of voriconazole, which prepares water-soluble linear polycyclodextrins, and then loads voriconazole into the molecular cage of the water-soluble linear polycyclodextrins, so as to solve the problem that the voriconazole is slightly soluble in water, and cannot be directly loaded into the crosslinked structure. The injectable hydrogel can be injected into the base of the vitreous body during a central vitrectomy procedure, and can gel in situ. However, this scheme is an injectable agent, which can only be applied to the vitreous cavity. The hydrogel is a lumpy solid with weak adhesion, which produces obvious foreign body sensation and friction when used on the ocular surface, and is not conducive to the uniform distribution of the drug on the ocular surface. It is not suitable for use on the ocular surface as an eye drop.

### SUMMARY OF THE PRESENT INVENTION

In response to poor solubility and permeability of conventional voriconazole eye drops and short retention time thereof at the ocular surface, an object of the present invention is to provide a preparation method of voriconazole hydrogel and eye drops using the same. The present invention synthesizes voriconazole (VCZ)-containing hydrogel by synthesizing polyaldehyde oligomer (PAO), using the PAO and amino-functionalized hyaluronic acid (AHA) as raw materials, and employing a Schiff base condensation reaction, which improves aqueous solubility, penetration, and ocular surface retention time of VCZ, and overcomes problems encountered in the clinical application of voriconazole.

Accordingly, in order to accomplish the above object, the present invention provides:
a preparation method of voriconazole hydrogel, comprising steps of:
adding 2,3-diaminopropionic acid hydrochloride (DAPA) and sodium hydroxide to deionized water, stirring until completely dissolved to produce a DAPA solution; dropping the DAPA solution into triformylbenzene (TFB) ethanol solution, and reacting at room temperature; then centrifugating, washing precipitation with ethanol, and freeze-drying in vacuum, so as to obtain a polyaldehyde oligomer (PAO); and
adding the PAO and VCZ to a first PBS and dispersing ultrasonically at 50-65° C. to completely dissolve the VCZ, so as to obtain a first dispersion; adding an AHA to a second PBS to obtain a second dispersion; mixing the first dispersion with the second dispersion and reacting at room temperature to obtain a voriconazole-loaded nano-enzymatic hydrogel composite (NHC).

The sodium hydroxide increases solubility and provides an alkaline environment for the reaction. Preferably, 579.6-708.4 mg of the DAPA and 780.0-820.0 mg of the sodium hydroxide are added per 50.0 mL of the deionized water for dissolving, so as to produce the DAPA solution.

Preferably, the DAPA solution is dropped into 315-385.0 mL of 1.43 mg/mL TFB ethanol solution.

Preferably, a centrifugation speed for separating PAO precipitate is 4000-6000 rpm, more preferably 5000 rpm.

Preferably, the preparation method further comprises: sequentially adding ethylenediamine hydrochloride, N-hydroxysuccinimide (NHS), 1-ethyl-(3-dimethylaminopropyl) carbodiimide (EDC) and 4-dimethylaminopyridine (DMAP) to a hyaluronic acid (HA) solution, adjusting a pH value to 4.8-5.2, and stirring at 25-28° C. for 1-2 days; then adjusting the pH value to 6.8-7.2, discarding unreacted components or by-products, and freeze-drying to obtain a white AHA.

Preferably, a concentration of the HA solution is 5.0 mg/mL, wherein 4.4-5.4 g of the ethylenediamine hydrochloride, 6.4-7.8 g of the NHS, 10.7-13.1 g of the EDC, and 4.5-5.5 mg of the DMAP are added per 90.0-110 mL of the HA solution.

Preferably, unreacted components or by-products are removed by dialysis during AHA preparation, with a molecular weight cut-off of 8000-14000 Da.

The present invention also provides a preparation method of voriconazole hydrogel containing nano-enzyme. The nano-enzyme is prepared by metal-phenol coordination of a soluble copper salt with a polyphenolic compound; the amino-functionalized hyaluronic acid and the nano-enzyme are added to the second PBS to obtain the second dispersion.

Preferably, the soluble copper salt is copper sulfate or copper chloride. The polyphenolic compound is selected from a group consisting of proanthocyanidin (PC), soy isoflavone, catechin, quercetin, anthocyanin, curcumin, and resveratrol.

Preferably, CuSO₄·5H₂O, the PC and deionized water are mixed and stirred to form a solution, a pH value of the solution is adjusted to 7.2-7.6; then the solution is heated to 50-55° C. for a 3-hour-reaction before centrifugation; precipitate is washed with deionized water, and is dried at 60-65° C.; with a preservation temperature of 2-4° C., a CuPC nano-enzyme is obtained.

Preferably, 1600.0-1900.0 mg of the CuSO₄·5H₂O and 50.0-58.0 mg of the PC are dissolved per 100.0 mL of the deionized water.

Preferably, a centrifugation speed for separating the CuPC nano-enzyme precipitate is 5000-7000 rpm, more preferably 6000 rpm.

Preferably, for the first dispersion, 500.0 mg of the PAO and 250.0 mg of the VCZ are added per 100.0 mL of the first PBS; for the second dispersion, 1500.0 mg of the AHA and 4.00 mg of the nano-enzyme (can be omitted) are added per 100.0 mL of the second PBS, the first dispersion and the second dispersion are mixed according to a volume ratio of 1:1.

The present invention also provides a preparation method of a topical ocular drug, comprising: applying a therapeutically effective amount the voriconazole hydrogel prepared by the above preparation, wherein the topical ocular drug is for mechanical injury or fungal keratitis.

Preferably, a causative agent of the fungal keratitis is Fusarium, Aspergillus, Candida spp., Penicillium spp. or yeast.

The present invention has the following beneficial effects:
(1) The present invention prepares PAO that promotes the dissolution of voriconazole, which solves the poor solubility of voriconazole.
(2) Drug permeability of the present invention is sufficient. Viscosity and ocular surface retention time are significantly increased. The concentration of voriconazole is only 0.25% (1/4 of the prior art), which can achieve better results. Moreover, the eye drops can be used only 2 times a day, reducing the frequency of medication.
(3) The NHC prepared by the present invention is an anionic hydrogel with thixotropic properties, which can be thinned during eyelid shear, which is conducive to drug redistribution and can reduce friction. As the same time, the hydrogel carries negative electricity, showing an electrostatic repulsion antifungal effect.
(4) The NHC prepared by the present invention is capable of scavenging ROS in both HCECs and HCFs.

### BRIEF DESCRIPTION OF THE DRAWINGS

FIG. 1 illustrates characterization of nano-enzyme particles prepared in an embodiment 3;

FIG. 2 is an FTIR diagram of an NHC prepared in the embodiment 3 and its precursors;

FIG. 3 is an SEM image of the NHC prepared in the embodiment 3;

FIG. 4 is a graph characterizing thixotropic properties of the NHC prepared in the embodiment 3;

FIG. 5 is a zeta potential diagram of the NHC prepared in the embodiment 3;

FIG. 6 illustrates evaluation of multi-enzyme mimetic activity of the NHC prepared in the embodiment 3;

FIG. 7 is a concentration-time profile of voriconazole in aqueous humor according to a test 7;

FIG. 8 is photographs of NHC treatment of corneal epithelial damage according to an animal experiment 1;

FIG. 9 is photographs of NHC treatment of fungal keratitis according to an animal experiment 2; and

FIG. 10 illustrates an inhibitory ring of the NHC prepared in the embodiment 1.

### DETAILED DESCRIPTION OF THE PREFERRED EMBODIMENT

The present invention will be further illustrated below in conjunction with specific embodiments and accompanying drawings.

Where specific techniques or conditions are not indicated in the embodiments, they are in accordance with the techniques or conditions described in common literatures in the art, or in accordance with product specifications. Where the manufacturer of the reagents or instruments used is not indicated, they are conventional and commercially available through regular channels.

### I. Material Preparation

### Embodiment 1

### (1) Preparation of Amino-Functionalized Hyaluronic Acid:

sequentially adding ethylenediamine hydrochloride (4.4 g), NHS (6.4 g), EDC (10.7 g) and 4.5 mg DMAP to a HA solution (5.0 mg/mL, 100.0 mL), adjusting a pH value to 5.0, and stirring at 25° C. for 1 day; then adjusting the pH value to 6.8; dialyzing with 8000-14000 Da cut-off dialysis tube for 2 days, thereby discarding unreacted components or by-products; and freeze-drying a mixture in the dialysis tube for 48hours to obtain a white AHA.

### (2) Preparation of Polyaldehyde Oligomer:

adding DAPA (579.6 mg) and sodium hydroxide (780.0 mg) to deionized water (50.0 mL), stirring at 1000 rpm for 30 min until completely dissolved; dropping the DAPA solution into 315.0 mL of 1.43 mg/mL TFB ethanol solution, and reacting at room temperature for 1 week; then centrifugating at 5000 rpm for 10 min, washing precipitation with ethanol for 3 times, and freeze-drying in vacuum for 48 hours.

(3) Preparation of NHC using Schiff Base Condensation Reaction adding the PAO (500.0 mg) and VCZ (250.0 mg) to a first PBS (100.0 mL) and dispersing ultrasonically at 55° C. for 30 min to completely dissolve the VCZ; adding the AHA (1500.0 mg) to a second PBS (100.0 mL), and shaking to disperse CuPC; mixing the above solutions according to a volume ratio of 1:1, and reacting at room temperature for 5 min to obtain the NHC.

### Embodiment 2

### (1) Preparation of Amino-Functionalized Hyaluronic Acid:

sequentially adding ethylenediamine hydrochloride (4.9 g), NHS (7.1 g), EDC (11.9 g) and 5.0 mg DMAP to a HA solution (5.0 mg/mL, 100.0 mL), adjusting a pH value to 5.0, and stirring at 25°° C. for 1 day; then adjusting the pH value to 7.0; dialyzing with 8000-14000 Da cut-off dialysis tube for 2 days, thereby discarding unreacted components or by-products; and freeze-drying a mixture in the dialysis tube for 48hours to obtain a white AHA.

### (2) Preparation of Polyaldehyde Oligomer:

adding DAPA (644.0 mg) and sodium hydroxide (800.0 mg) to deionized water (50.0 mL), stirring at 1000 rpm for 30 min until completely dissolved; dropping the DAPA solution into 350.0 mL of 1.43 mg/mL TFB ethanol solution, and reacting at room temperature for 1 week; then centrifugating at 5000 rpm for 10 min, washing precipitation with ethanol for 3 times, and freeze-drying in vacuum for 48 hours.

(3) Preparation of NHC using Schiff Base Condensation Reaction adding the PAO (500.0 mg) and VCZ (250.0 mg) to a first PBS (100.0 mL) and dispersing ultrasonically at 65° C. for 30 min to completely dissolve the VCZ;

adding the AHA (1500.0 mg) and CuPC (4.00 mg) to a second PBS (100.0 mL), and shaking to disperse CuPC; mixing the above solutions according to a volume ratio of 1:1, and reacting at room temperature for 5 min to obtain the NHC.

### Embodiment 3

### (1) Preparation of CuPC Nano-Enzyme by Metal-Phenol Coordination:

adding CuSO4.5H2O (1750.0 mg), PC (54.0 mg) and deionized water (100.0 mL) to a round bottom flask, and stirring a mixture at 600 rpm for 5 min to form a homogeneous solution; using 1.0 M NaOH to adjust a pH value of the solution to 7.4;then heating to 50°° C. and keeping for 3 hours; centrifugating at 6000 rpm for 10 min; washing precipitate with deionized water for 3 times, and drying at 60° C. overnight; preserving at 4° C. for subsequent use.

(2) Preparation of amino-functionalized hyaluronic acid, same as the embodiment 2.

(3) Preparation of polyaldehyde oligomer, same as the embodiment 2.

(4) Preparation of NHC using Schiff base condensation reaction adding the PAO (500.0 mg) and VCZ (250.0 mg) to a first PBS (100.0 mL) and dispersing ultrasonically at 65° C. for 30 min to completely dissolve the VCZ; adding the AHA (1500.0 mg) and CuPC (4.00 mg) to a second PBS (100.0 mL), and shaking to disperse CuPC; mixing the above solutions according to a volume ratio of 1:1, and reacting at room temperature for 5 min to obtain the NHC.

### Embodiment 4

Copper chloride (941.0 mg) is used instead of the CuSO4.5H2O to prepare the CuPC nano-enzyme, and other steps are the same as the embodiment 3.

### Embodiment 5

Tannins (127.0 mg) is used instead of the proanthocyanidin to prepare the CuPC nano-enzyme, and other steps are the same as the embodiment 3.

### II. Properties of Materials

Physical properties, multi-enzyme mimetic activity, in vitro/in vivo release, in vitro/in vivo biocompatibility and in vitro antifungal activity are evaluated.

### Test 1

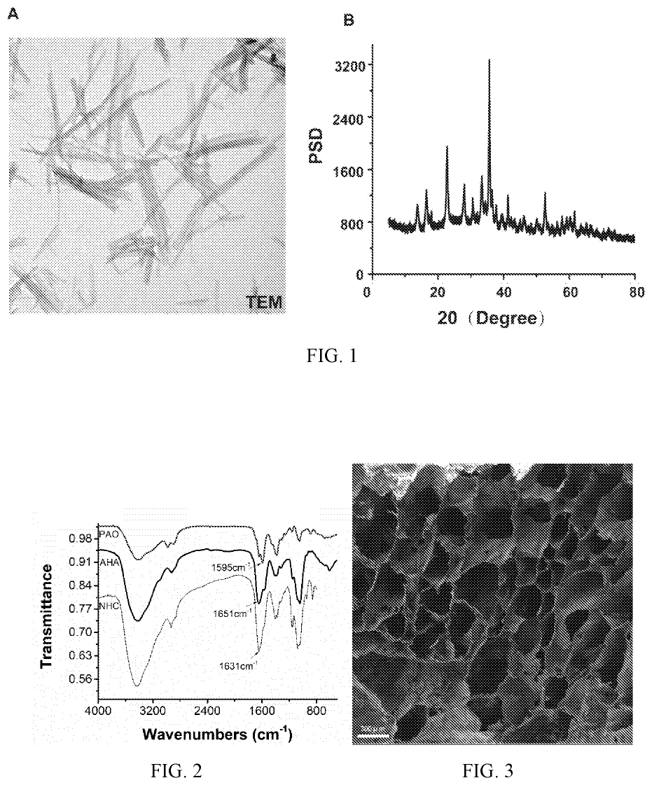

The physical properties of CuPC obtained in the embodiment 3 was characterized. Referring to FIG. 1, (A) is TEM image showing a rod-like morphology of CuPC, with lengths ranging from tens to hundreds of nanometers; and (B) is XRD image showing a crystal structure of CuPC. XRD diffraction peaks of CuPC were located at 13.8, 16.5, 22.8, 28.0, 30.6, 33.4, 35.6, 41.3, and 52.7, which were in correspondence with those of the Cu-tannic acid-liganded nanosheets.

### Test 2

The preparative precursors AHA, PAO, and NHC of the hydrogel prepared in the embodiment 3 were subjected to Fourier Transform Infrared Spectroscopy, as shown in FIG. 2.

### Test 3

The NHC obtained in the embodiment 3 was freeze-dried and tested by scanning electron microscopy for observation, and test results are shown in FIG. 3. The NHC has a large pore size, which is conducive to the maintenance of high water content, permeability, and the exchange of nutrients.

### Test 4

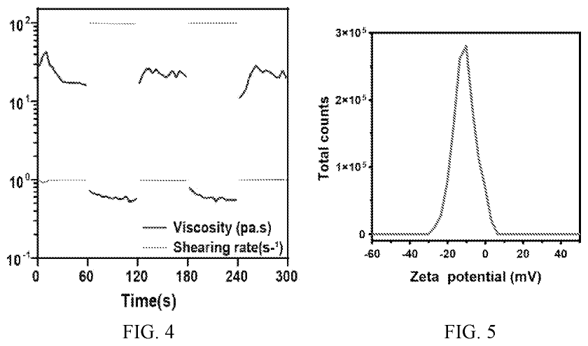

The NHC obtained in the embodiment 3 was subjected to a rheological property test to observe its thixotropic function. Referring to FIG. 4, the NHC has shear-thinning and thixotropic properties.

### Test 5

The NHC obtained in the embodiment 3 was subjected to potentiometric testing, and it was observed that the anionic hydrogel carried negative charges, as shown in FIG. 5.

### Test 6

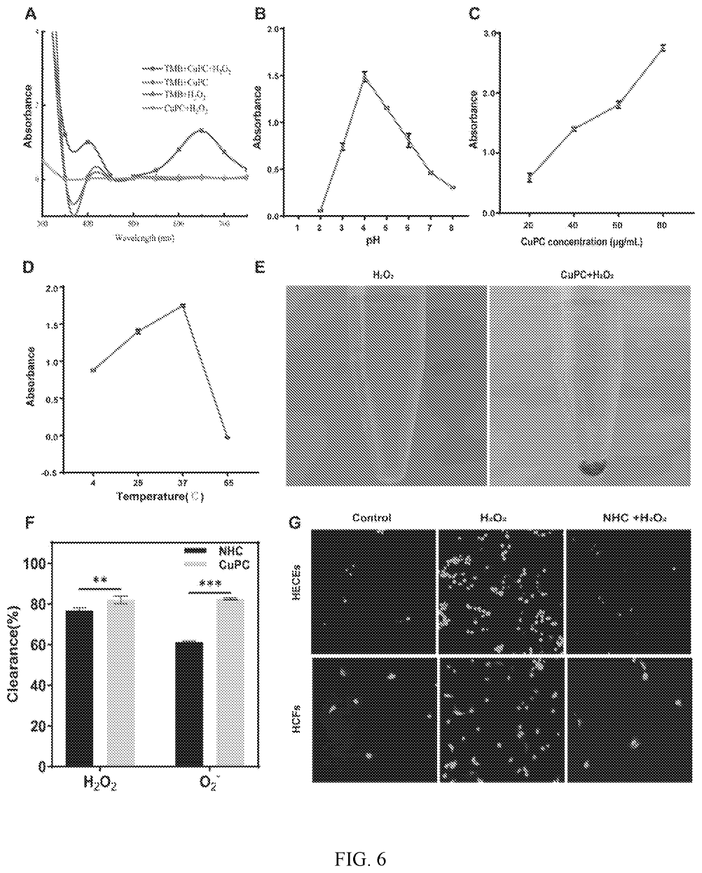

The NHC obtained in the embodiment 3 was tested for progressive scavenging of ROS, and it was observed that the nanocomposite hydrogel possessed peroxidase activity, catalase activity, and superoxide dismutase activity. Referring to FIG. 6, (A) is absorption spectra of the reaction systems with different compositions; (B) indicates the pH value, (C) illustrates CuPC concentration, (D) shows the effect of reaction temperature on the peroxidase mimetic activity of CuPC; (E) is optical photographs showing the interaction of CuPC with H₂O₂ to produce oxygen. (F) indicates removal capacities of CuPC and NHC for H₂O₂ and O₂⁻; and (G) is DCFH-DA staining diagram showing the ability of NHC to scavenge ROS in both HCECs and HCFs.

### Test 7

The concentration of voriconazole was measured by extracting the aqueous humor at different times after applying the NHC obtained in the embodiment 3 to New

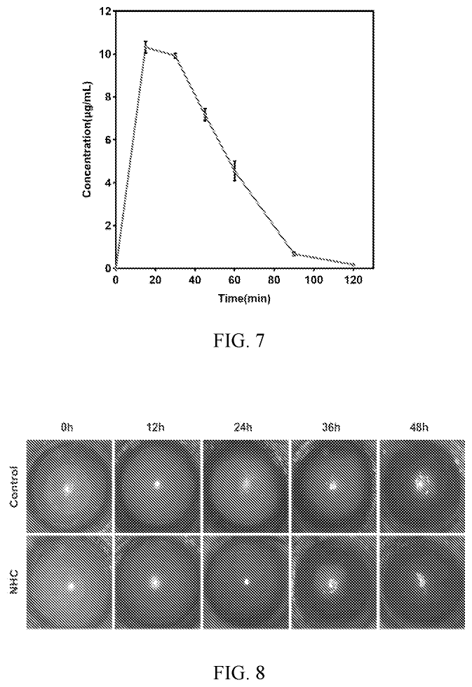

Zealand big white rabbits, and it was observed that the nano-enzymatic composite hydrogel improved the permeability of the drug, as shown in FIG.7.

### Test

Related literature reported that the peak concentration of clinical 1% voriconazole after 1 ocular medication given to New Zealand big white rabbits was 3.56±1 μg/mL, while the peak concentration of NHC after 1 ocular medication was 10.31±0.27 μg/mL as shown in FIG. 7, which is more than twice the effect of the clinical 1% voriconazole.

### III. Animal Experimentation

### Animal Experiment 1

C57BL/6 female mice were anesthetized by intraperitoneal injection of 0.06% pentobarbital sodium. The corneal epithelium in the center of the cornea was marked with a 2.5 mm ring-drill, and the corneal epithelium was scraped to prepare a corneal epithelial damage animal model.

The NHC prepared in the embodiment 1 was spotted on the cornea of the mice twice a day, and corneal epithelial repair conditions were observed and photographed at different times. The control group was given saline to spot the eyes twice a day, and the experimental group was given NHC to spot the eyes twice a day. As shown in FIG. 8, the corneal epithelial damage in the experimental group was significantly repaired after 36 hours, and the corneal epithelial damage was basically repaired after 48 hours. The repair speed of the control group was significantly slower than that of the experimental group, and part of the corneal epithelial damage area was not repaired after 48 hours. This indicates that NHC has obvious repairing effect on corneal epithelial mechanical damage.

### Animal Experiment 2

C57BL/6 female mice were anesthetized by intraperitoneal injection of 0.06% pentobarbital sodium. The corneal epithelium in the center of the cornea was marked with a 2.5 mm ring-drill, and the corneal epithelium was scraped off. An animal model of fungal keratitis was prepared by taking a piece of filter paper, soaking the center of the cornea with a suspension of Fusarium lycopersicum fungal spores (10⁷ CFU/mL), and closing the eyelid with 7-0 sutures.

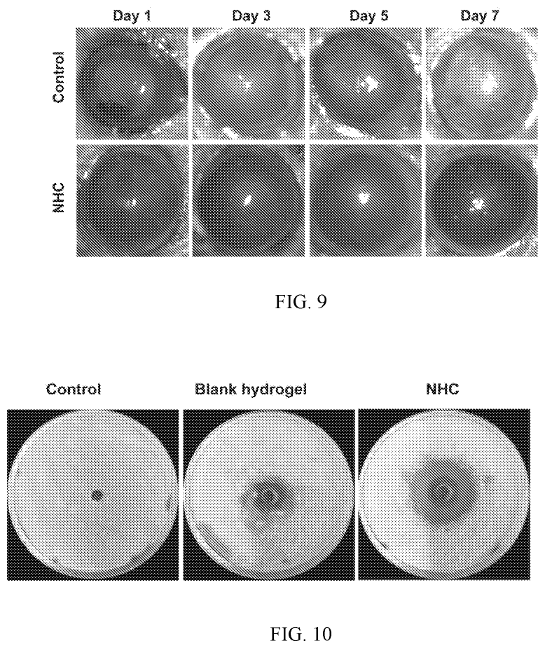

The NHC prepared in the embodiment 1 was spotted on the cornea of the mice twice a day, and treatment conditions of fungal keratitis were observed and photographed at different times. Saline was substituted for NHC in the control group. As shown in FIG. 9, on the 3rd day, the symptoms of fungal keratitis were significantly reduced, and the symptoms of fungal keratitis disappeared on the 7th day, which indicated that the NHC had a significant therapeutic effect on fungal keratitis.

The above-described embodiments are only preferred embodiments of the present invention, and are not intended to be limiting. Without departing from the spirit of the design of the present invention, various technical scheme deformations and improvements made by those of ordinary skill in the art shall fall within the protection scope as determined by the claims of the present invention.

What is claimed is:

1. A preparation method of voriconazole hydrogel, comprising steps of:
adding 2,3-diaminopropionic acid hydrochloride (DAPA) and sodium hydroxide to deionized water, stirring until completely dissolved to produce a DAPA solution; dropping the DAPA solution into triformylbenzene ethanol solution, and reacting at room temperature; then centrifugating, washing precipitation with ethanol, and freeze-drying in vacuum, so as to obtain a polyaldehyde oligomer;
sequentially adding ethylenediamine hydrochloride, N-hydroxysuccinimide, 1-ethyl-(3-dimethylaminopropyl) carbodiimide and 4-dimethylaminopyridine to a hyaluronic acid solution, adjusting a pH value to 4.8-5.2, and stirring at 25-28° C. for 1-2 days; then adjusting the pH value to 6.8-7.2, discarding unreacted components or by-products, and freeze-drying to obtain a white amine-functionalized hyaluronic acid; and
adding the polyaldehyde oligomer and voriconazole to a first phosphate-buffered saline (PBS) and dispersing ultrasonically at 50-65° C. to completely dissolve the voriconazole, so as to obtain a first dispersion; adding the amino-functionalized hyaluronic acid to a second PBS to obtain a second dispersion; mixing the first dispersion with the second dispersion and reacting at room temperature to obtain the voriconazole hydrogel.

2. The preparation method, as recited in claim 1, wherein 579.6-708.4 mg of the 2,3-diaminopropionic acid hydrochloride and 780.0-820.0 mg of the sodium hydroxide are added per 50.0 mL of the deionized water for dissolving, so as to produce the DAPA solution; and then the DAPA solution is dropped into 315-385.0 mL of 1.43 mg/mL triformylbenzene ethanol solution.

3. The preparation method, as recited in claim 1, wherein the second dispersion further comprises a nano-enzyme which is prepared by metal-phenol coordination of a soluble copper salt with a polyphenolic compound; the amino-functionalized hyaluronic acid and the nano-enzyme are added to the second PBS to obtain the second dispersion; wherein the polyphenolic compound is proanthocyanidin.

4. The preparation method, as recited in claim 3, wherein the soluble copper salt is copper sulfate or copper chloride.

5. The preparation method, as recited in claim 4, wherein CuSO₄·5H₂O, the proanthocyanidin and deionized water are mixed and stirred to form a solution, a pH value of the solution is adjusted to 7.2-7.6; then the solution is heated to 50-55° C. for reacting before centrifugation; precipitate is washed with deionized water, and is dried at 60-65° C.; with a preservation temperature of 2-4° C., a CuPC nano-enzyme is obtained.

6. The preparation method, as recited in claim 3, wherein for the first dispersion, 500.0 mg of the polyaldehyde oligomer and 250.0 mg of the voriconazole are added per 100.0 mL of the first PBS; for the second dispersion, 1500.0 mg of the amino-functionalized hyaluronic acid and 4.00 mg of the nano-enzyme are added per 100.0 mL of the second PBS, the first dispersion and the second dispersion are mixed according to a volume ratio of 1:1.

7. The preparation method, as recited in claim 4, wherein for the first dispersion, 500.0 mg of the polyaldehyde oligomer and 250.0 mg of the voriconazole are added per 100.0 mL of the first PBS; for the second dispersion, 1500.0 mg of the amino-functionalized hyaluronic acid and 4.00 mg of the nano-enzyme are added per 100.0 mL of the second PBS, the first dispersion and the second dispersion are mixed according to a volume ratio of 1:1.

8. The preparation method, as recited in claim 5, wherein for the first dispersion, 500.0 mg of the polyaldehyde oligomer and 250.0 mg of the voriconazole are added per 100.0 mL of the first PBS; for the second dispersion, 1500.0 mg of the amino-functionalized hyaluronic acid and 4.00 mg of the nano-enzyme are added per 100.0 mL of the second PBS, the first dispersion and the second dispersion are mixed according to a volume ratio of 1:1.

9. A preparation method of a topical ocular drug, comprising: applying a therapeutically effective amount the voriconazole hydrogel prepared by the preparation method as recited in claim 1.

10. The preparation method, as recited in claim 9., wherein the topical ocular drug is for mechanical injury or fungal keratitis.

11. The preparation method, as recited in claim 10, wherein a causative agent of the fungal keratitis is Fusarium, Aspergillus, Candida spp., Penicillium spp. or yeast.
